(19) United States
(12) Patent Application Publication
(10) Pub. No.: US 2025/0073403 A1
(43) Pub. Date: Mar. 6, 2025

(54) SLEEP PERFORMANCE SCORING DURING THERAPY

(71) Applicant: ResMed Sensor Technologies Limited, Dublin (IE)

(72) Inventors: Hannah Meriel Kilroy, Dublin (IE); Redmond Shouldice, Dublin (IE); Michael John Costello, Dublin (IE)

(21) Appl. No.: 18/034,543

(22) PCT Filed: Oct. 28, 2021
(86) PCT No.: PCT/IB2021/059996

§ 371 Date: Apr. 28, 2023

Related U.S. Application Data
(60) Provisional application No. 63/107,935, filed on Oct. 30, 2020.

(51) Int. Cl. A61M 16/00; A61M 16/06

(52) U.S. Cl. CPC A61M 16/0003; A61M 16/024; A61M 16/0627; A61M 2205/15; A61M 2205/3303

(57) ABSTRACT

Sleep performance scores can be generated for an individual receiving respiratory therapy. Sensor data can be obtained from one or more sensors while the user is sleeping and using a respiratory therapy system. The sensor data can be used to determine one or more usage variables associated with use of the respiratory therapy system, as well as sleep stage information indicative of the stages of sleep undergone by the user while sleeping. A sleep performance score can be calculated using the one or more usage variables and the sleep stage information. In some cases, the sleep stage information can be used to apply weightings to one, some, or all of the one or more usage variables. The sleep performance score can indicate compliance, efficacy, quality, and/or general use of the respiratory therapy system, taking into account the relationship between sleep stage and use of the respiratory therapy system.

### CROSS REFERENCE TO RELATED APPLICATIONS

The present application claims the benefit of U.S. Provisional Patent Application No. 63/107,935 filed Oct. 30, 2020 and entitled “SLEEP PERFORMANCE SCORING DURING THERAPY,” the disclosure of which is hereby incorporated by reference in its entirety.

### TECHNICAL FIELD

The present disclosure relates to treatment of sleep conditions generally and more specifically to providing useful metrics to score sleep performance during treatment of sleep conditions.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), chest wall disorders, and insomnia. The sleep-related respiratory disorders can be associated with one or more events that may occur during sleep, such as, for example, snoring, an apnea, a hypopnea, a restless leg, a sleeping disorder, choking, an increased heart rate, labored breathing, an asthma attack, an epileptic episode, a seizure, or any combination thereof. Individuals suffering from such sleep-related respiratory disorders are often treated using one or more medical devices to improve sleep and reduce the likelihood of events occurring during sleep. An example of such a device is a respiratory therapy system that can provide positive airway pressure to the individual, although other devices may be used. There is a need to provide meaningful metrics regarding use of such devices, such as to monitor compliance, increase user engagement, monitor efficacy of treatment, and the like.

### SUMMARY

Certain aspects of the present disclosure include a method for scoring sleep performance, the method comprising: receiving sensor data from one or more sensors, the sensor data being associated with a sleep session of a user using a respiratory therapy system; determining, from the received sensor data, one or more usage variables associated with use of the respiratory therapy system; determining, from the received sensor data, sleep stage information associated with the sleep session; and calculating a sleep performance score for the sleep session using the determined one or more usage variables and the sleep stage information.

Certain aspects of the present disclosure include a system comprising: a control system including one or more processors; and a memory having stored thereon machine readable instructions; wherein the control system is coupled to the memory, and the method described above is implemented when the machine executable instructions in the memory are executed by at least one of the one or more processors of the control system.

Certain aspects of the present disclosure include a system for scoring sleep performance, the system including a control system configured to implement the method described above.

Certain aspects of the present disclosure include a computer program product comprising instructions which, when executed by a computer, cause the computer to carry out the method disclosed above. In some cases, the computer program product is a non-transitory computer readable medium.

### BRIEF DESCRIPTION OF THE DRAWINGS

The specification makes reference to the following appended figures, in which use of like reference numerals in different figures is intended to illustrate like or analogous components.

FIG. 1 is a functional block diagram of a system for scoring sleep performance, according to certain aspects of the present disclosure.

FIG. 2 is a perspective view of the system of FIG. 1, a user, and a bed partner, according to certain aspects of the present disclosure.

FIG. 3 illustrates an example timeline for a sleep session, according to certain aspects of the present disclosure.

FIG. 4 illustrates an example hypnogram associated with the sleep session of FIG. 3, according to certain aspects of the present disclosure.

FIG. 5 is a chart illustrating usage variables associated with the hypnogram of FIG. 4, according to certain aspects of the present disclosure.

FIG. 6 is a flowchart depicting a process for scoring sleep performance, according to certain aspects of the present disclosure.

FIG. 7 is a flowchart depicting a process for scoring sleep performance using acclimatization stages, according to certain aspects of the present disclosure.

FIG. 8 is a chart illustrating a user's progress through acclimatization stages, according to certain aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Certain aspects and features of the present disclosure relate to systems and methods for generating sleep performance scores for an individual making use of a respiratory therapy system (e.g., using a respiratory therapy system to provide respiratory therapy during a sleep session). A system can obtain sensor data from one or more sensors while the user engages in a sleep session and makes use of the respiratory therapy system. Sensor data can be used to determine one or more usage variables associated with use of the respiratory therapy system, as well as sleep stage information indicative of the stages of sleep and/or sleep state (e.g., awake or asleep) undergone by the user during the sleep session. A sleep performance score can be calculated using the one or more usage variables and the sleep stage information. In some cases, the sleep stage information can be used to apply weightings to one, some, or all of the one or more usage variables. The sleep performance score can be used to indicate the compliance, efficacy, quality, and/or general use of the respiratory therapy system, taking into account the relationship between sleep stage and use of the respiratory therapy system.

Certain aspects of the present disclosure are useful to generate a sleep performance score associated with a sleep session of a user receiving respiratory therapy. Respiratory therapy can be applied using a respiratory therapy device, such as a respiratory device that supplies pressurized air to the user via a conduit and user interface. While receiving the respiratory therapy, the user can engage in a sleep session, during which sensor data can be collected from one or more sensors, such as sensors in the respiratory therapy device, sensors in a user device (e.g., smartphone), sensors in an activity tracker (e.g., wearable activity tracker), or other sensors located in, on, or around the user (e.g., implantable devices, clothing-integrated sensors, mattress-integrated sensors, wall-mounted or ceiling-mounted sensors, or the like). The data collected from the one or more sensors can be used to determine one or more usage variables associated with use of the respiratory therapy system, as well as sleep stage information. Other variables and/or information can be determined using the sensor data.

Usage variables associated with use of the respiratory therapy system can include any suitable variable related to how a user makes use of the respiratory therapy system. Examples of suitable usage variables include usage time (e.g., a duration of time the user makes use of the respiratory therapy system); a seal quality variable (e.g., an indication of the quality of seal between the user and the user interface); a leak flow rate variable (e.g., an indication of the rate of flow of unintentional leaks, such as leaks through a poor-quality seal or mouth-breathing while wearing a nasal pillow type user interface); event information (e.g., an indication of detected events that occurred during the sleep session, such as an apnea-hypopnea index (AHI)); user interface compliance information (e.g., an indication of detected user interface transition events, such as donning or removing the user interface); a number of therapy sub-sessions within the sleep sessions (e.g., a number of separate blocks of continuous usage of the respiratory therapy system); and user interface pressure. Other usage variables can be used. Statistical summaries (e.g., averages, maximums, minimums, counts, and the like) of one or more usage variables can be used as one or more additional usage variables. The one or more usage variables can include any suitable combination of usage variables.

Determining a usage variable can include processing sensor data to identify one or more values associated with the usage variable. The one or more values can be a measurement or calculated score associated with the usage variable. For example, a seal quality variable can be a measurement of leak flow rate (e.g., in L/min) or a seal quality score (e.g., 18 out of 20).

Determining a usage variable can include determining a single value or multiple values (e.g., timestamped values). For example, in some cases, determining a seal quality variable can include determining a single value representative of the overall (e.g., average) seal quality throughout the sleep session (e.g., 18 out of 20). In some cases, however, determining a seal quality variable can include determining a set of timestamped values representative of the seal quality over time (e.g., on a scale of 0 to 20, 18 at 10:00:00 PM, 18.1 at 10:00:05 PM, 18.2 at 10:00:10 PM, and the like), such as data that can be charted to depict seal quality throughout a duration of time.

Sleep stage information can include information indicative of the sleep stages undergone by the user during the sleep session. Examples of sleep stages include a wakefulness stage, a rapid eye movement (REM) stage, a light sleep stage, and a deep sleep stage. The sensor data can be processed to determine times when the user enters and exits various stages of sleep. In some cases, determining sleep stage information can include determining a total duration of time the user spent in each sleep stage. In an example 8-hour sleep session, the sleep stage information may indicate a total of 21 minutes in wakefulness, 101 minutes in REM sleep, 267 minutes in light sleep, and 91 minutes in deep sleep. In some cases, however, determining sleep stage information can include generating timestamped data indicative of the sleep stage of the user at various times throughout the sleep session, such as data that can be charted to generate a hypnogram of the user's sleep session.

While the usage variables are indicative of use of the respiratory therapy system, a score based solely on usage variables may not be as informative and useful as a score that is based on usage variables and sleep stage information. For example, it can be informative and useful to track a total amount of time a user makes use of a respiratory therapy device during a sleep session. Generally, the more time used, the better. Using a respiratory therapy device for only the first couple hours of a sleep session may be undesirable. Thus, it can be useful to provide a score to a user that increases (e.g., improves) the longer the user makes use of the respiratory therapy device. A simple score based on only usage variables does just that, indicating higher values for longer usage times and lower values for shorter usage times. While such a simple score may be useful to encourage the user to use the respiratory therapy device for longer periods of time, it may also cause undesirable behavior or not reflect important details about how the respiratory therapy device is actually used. For example, a user may achieve a high-value simple score by simply using the respiratory therapy device for a longer period of time prior to falling asleep, although this high-value may be counterproductive, since it does not necessarily reflect the user receiving any substantial benefit from using the respiratory therapy device while awake. However, a sleep performance score calculated based on both usage variables and sleep stage information can provide a more informative and useful score. Since apnea and hypopnea events may be more prevalent (e.g., because of decreased tone of the genioglossus muscle in the tongue) during REM sleep and more detrimental (e.g., due to the chance of interrupting REM sleep, negatively impacting spatial memory, and/or reducing amount of deep sleep) during REM and deep sleep, it may be more useful to track an amount of time the respiratory therapy device is used during REM sleep and/or during deep sleep. Thus, in addition to tracking overall usage time, the amount of time the respiratory therapy device is used in certain sleep stages (e.g., REM sleep or deep sleep) can be emphasized (e.g., weighted more strongly) than time the respiratory therapy device is used in other sleep stages (e.g., awake or light sleep). Thus, even if a user makes use of a respiratory therapy device for longer periods of time before falling asleep, the sleep performance score may not increase much or at all. However, if the same user makes use of the respiratory therapy device for longer periods of REM stage sleep, the sleep performance score may increase substantially.

Likewise, detection of apnea or hypopnea events can be an informative and useful variable to track, but the prevalence of detected (e.g., apparent) events during wakefulness may be a false-detection, which can be discounted, and the prevalence of detected events during REM sleep may be an indication that the respiratory therapy device is not providing sufficient respiratory therapy. Thus, detected events associated with REM sleep stages may be emphasized over detected events associated with other sleep stages, such as wakefulness.

Likewise, a seal quality variable or a leak flow rate can be an informative and useful variable to track. Since poor seals and unintentional leaks can increase the risk of an apnea or hypopnea event, drops in seal quality variables or leak flow rates can be indicative of a risk of an event occurring. Thus, the prevalence of a poor seal or an unintentional leak during REM sleep, when an event could have a substantially detrimental effect (e.g., interrupting REM sleep, negatively impacting spatial memory, and/or reducing amount of deep sleep), may be more important than the prevalence of a poor seal or an unintentional leak during wakefulness. Thus, low seal quality variables or low leak flow rates associated with REM sleep stages may be emphasized over low seal quality variables or low leak flow rates associated with wakefulness. Additionally, poor seals and unintentional leaks that are associated with light sleep may be detrimental because of the risk of impacting user experience since the user may be more conscious during light sleep, which can affect user compliance. For example, a poor seal during light sleep may be perceived by, and be uncomfortable for, the user and may lead to a user removing the user interface. Thus, low seal quality variables or low leak flow rates associated with light sleep stages may be emphasized over low seal quality variables or low leak flow rates associated with deep sleep stages.

Therefore, a sleep performance score that is based on usage variable(s) and sleep stage information can be especially useful and informative. Calculating such a sleep performance score can also include using other data in addition to using the usage variable(s) and the sleep stage information. In some cases, calculating sleep performance score can include applying a weighting value to each of the usage variable(s), which weighting value can be adjusted (or generated) based at least in part on the sleep stage information and/or at least in part on another usage variable. In some cases, weighting values can be adjusted (or generated) based at least in part on sleep-related parameters, such as a total time in bed, a total sleep time, a sleep onset latency, a wake-after-sleep-onset parameter, a sleep efficiency, a fragmentation index, or any combination thereof.

In an example, where a sleep performance score is calculated based on usage variables that are each single values (e.g., averages or scores associated with an entire sleep session), the usage variables can include a usage time (U), a seal quality variable (Q), event information (E), and user interface compliance information (C), and the sleep performance score (Score) may be calculated according to the following equation.

$$Score = {χ}_{1} U + {χ}_{2} Q + {χ}_{3} E + {χ}_{4} C$$

In this example, χ₁ is a weighting value associated with usage time, χ₂ is a weighting value associated with seal quality, χ₃ is a weighting value associated with event information, and χ₄ is a weighting value associated with interface compliance information. Each of these weighting values can be determined based on the sleep stage information, other usage variables, or a combination thereof. Thus, depending on the time spent in different sleep stages, the weighting values may be adjusted.

In an example, on a first night with a relatively high amount of time spent in REM sleep, χ₃ may be higher than on nights with relatively low amounts of time spent in REM sleep. Such changes in weighting of χ₃ can emphasize that events occurring when the user is otherwise engaging in good REM sleep can be more detrimental, and thus affect the sleep performance score more, than events occurring when the user is otherwise not engaging in good REM sleep. Other examples can be used.

In another example, on a first night with a relatively high seal quality variable, χ₄ may be higher than on nights with relatively low seal quality variable. Such changes in weighting of χ₄ can emphasize that on nights where the seal quality is bad, the user may be more likely to take the user interface off to reposition the user interface, and thus the impact on the overall sleep performance score should not be as significant as on nights where the seal quality is good and the user is removing the user interface for other reasons.

In another example, where a sleep performance score is calculated based on usage variables that are time-dependent values (e.g., timestamped values over the course of the sleep session), the usage variables can be functions of time and can include a usage time (U(t)), a seal quality variable (Q(t)), event information (E(t)), and user interface compliance information (C(t)), and the sleep performance score (Score) may be calculated according to the following equation.

$$Score = {χ}_{1} U ( t ) + {χ}_{2} Q ( t ) + {χ}_{3} E ( t ) + {χ}_{4} C ( t )$$

In this example, χ₁ is a weighting value associated with usage time, χ₂ is a weighting value associated with seal quality, χ₃ is a weighting value associated with event information, and χ₄ is a weighting value associated with interface compliance information.

In another example, where a sleep performance score is calculated based on usage variables that are time-dependent values (e.g., timestamped values over the course of the sleep session), the usage variables can be functions of time and can include a usage time (U(t)), a seal quality variable (Q(t)), event information (E(t)), and user interface compliance information (C(t)), and the sleep performance score (Score) may be calculated according to the following equation.

$$Score = {χ}_{1} ( t ) U ( t ) + {χ}_{2} ( t ) Q ( t ) + {χ}_{3} ( t ) E ( t ) + {χ}_{4} ( t ) C ( t )$$

In this example, the weighting values are time-dependent, where χ₁(t) is a weighting value associated with usage time, χ₂(t) is a weighting value associated with seal quality, χ₃(t) is a weighting value associated with event information, and χ₄(t) is a weighting value associated with interface compliance information.

In yet another example, sleep performance score can be calculated based on segmented usage variables. The usage variables can be segmented by sleep stage, using the sleep stage information. For example, a total usage time (U) can be segmented into usage time segments using the sleep stage information, including usage time during wakefulness (U_{W}), usage time during REM sleep (U_{R}), usage time during light sleep (U_{L}), and usage time during deep sleep (U_{D}). Similar segmentation can be performed on any usage variables (e.g., seal quality segments, air leek segments, detected event segments, user interface compliance segments). While the sleep performance score may be calculated using a number of usage variables that are segmented, in an example with only a single usage variable that is usage time, sleep performance score (Score) may be calculated according to the following equation.

$$Score = {χ}_{1 W} {U}_{W} + {χ}_{1 R} {U}_{R} + {χ}_{1 L} {U}_{L} + {χ}_{1 D} {U}_{D}$$

In this example, χ_{1W} is a weighting value associated with usage time during wakefulness, χ_{1R} is a weighting value associated with usage time during REM sleep, χ_{1L} is a weighting value associated with usage time during light sleep, and χ_{1D} is a weighting value associated with usage time during deep sleep. In some cases, the aforementioned usage variable(s) and/or weighting values can be time-dependent.

In an example of the segmented usage variables, because apnea events are more prevalent in REM sleep due to the decreased tone of the genioglossus muscle in the tongue, respiratory therapy may be more important while the user is in REM sleep than while the user is awake or in light sleep. Thus, the weighting values can be set appropriately, with χ_{1R} being larger than χ_{1W} and χ_{1L} (e.g., giving greater score increases for using the respiratory therapy device for a certain duration of time in REM sleep, while giving lower score increases for using the respiratory therapy device for the same duration of time in wakefulness or light sleep).

In yet another example, sleep performance score can be calculated based on segmented usage variables that are segmented based on another usage variable. For example, user interface compliance information (C) can be segmented into user interface compliance segments using a seal quality variable, including user interface compliance information when the seal quality variable is low (C_{L}) and user interface compliance information when the seal quality variable is high (C_{H}). Similar segmentation can be performed on any usage variables. While the sleep performance score may be calculated using a number of usage variables that are segmented, in an example with only a single usage variable that is user interface compliance information, sleep performance score (Score) may be calculated according to the following equation.

$$Score = {χ}_{1 L} {C}_{L} + {χ}_{1 H} {C}_{H}$$

In this example, χ_{1L} is a weighting value associated with user interface compliance when the seal quality variable is low (e.g., below a threshold value) and χ_{1H} is a weighting value associated with user interface compliance when the seal quality variable is high (e.g., at or above a threshold value). In some cases, the aforementioned usage variable(s) and/or weighting values can be time-dependent. In the above example, χ_{1L} may be smaller than χ_{1H} so as to emphasize that detected user interface transitions while the seal quality variable is low (e.g., likely to be indicative of a user manipulating the user interface to improve seal quality) should not affect the overall sleep performance score as much as detected user interface transitions while the seal quality variable is high (e.g., likely to be an undesirable user interface transition).

Various schemes of applying weighting values to usage variables to determine sleep performance scores are disclosed above, such as with reference to the equations presented above.

In some cases, one, some, or all of the various schemes described herein can be combined to calculate a sleep performance score. For example, in some cases, a sleep performance score can include applying, to a usage variable, weighting values that are based on sleep stage information as well as applying weighting values that are based on another usage variable. In another example, a sleep performance score may be calculated by applying weighting values based on sleep stage information to a first usage variable while applying no weighting values (or a neutral weighting value) to a second usage variable.

As used herein, applying a weighting value to each of the usage variables is intended to be inclusive of applying a weighting value to fewer than all of the usage variables, in which case any usage variables to which no weighting value is applied can be considered to have a neutral weighting value (e.g., 1.0× or 100%) applied thereto. For example, applying a 0.75× weighting value to only a first usage variable out of a set of four usage variables and not applying any weighting values to the other usage variables is equivalent to applying the 0.75× weighting value to the first usage variable and applying a 1.0× weighting value to the remaining usage variables.

In some cases, weighting values described herein can be static weighting values that are stored in a memory accessible to the system calculating the sleep performance score. For example, the weighting value for usage time in REM sleep may always be 1.25× (or 125%). In some cases, however, weighting values can be dynamic, such as a function of certain data (e.g., another usage variable or sleep stage information) or an output from a machine learning algorithm (e.g., a deep neural network) trained to output weighting values from input data (e.g., sensor data, usage variable(s), or sleep stage information) to achieve an accurate sleep performance score (e.g., an objectively accurate or subjectively accurate score).

In some cases, a sleep quality score can be determined. The sleep quality score can be an indication of the quality of sleep undergone by the user during the sleep session. For example, a sleep session with many awakenings or interruptions may have a low sleep quality score, whereas a sleep session with fewer awakenings or interruptions may have a higher sleep quality score.

In some cases, the sleep quality score can be based on subjective feedback (e.g., feedback from a user indicating a subjective feeling of restfulness following a sleep session), can be based on objective data, or a combination of the two. Subjective feedback can include a user's rating of the user's sleep session and/or PROMS (patient reported outcome metrics) data collected from the user by a healthcare provider. In some cases, the subjective feedback can include subjective reasons about why the user feels the way they do about their sleep quality and/or the quality of the therapy they received. Such reasons can be stored, and optionally presented, in association with the sleep quality score and/or the sleep performance score.

In some cases, sleep quality score can be used in the calculation of the sleep performance score. In some cases, sleep quality score can be a component of the sleep performance score. In some cases, the sleep quality score can be used to determine weighting values to be applied to different components of the sleep performance score (e.g., weighting values applied to one or more usage variables). In some cases, such as when subjective feedback is collected, the subjective feedback can be used to directly modify one or more components of the sleep performance score or the sleep performance score itself, such as by incorporating (e.g., directly adding) a modification value instead of or in addition to affecting weighting values. The modification value can be a preset value selected based on the subjective feedback (e.g., “5” for a positive feedback or “−5” for a negative feedback), or can be a variable value based on the subjective feedback.

In an example, sleep quality score or a component thereof can be determined objectively, such as based on sleep stage information. In an example, time spent in different sleep stages can be used to determine a sleep quality score. Additionally or alternatively, the pattern of sleep stages (e.g., the sleep architecture) can be used to determine a sleep quality score. The sleep stage information can be segmented into sleep stage segments indicative of time spent in each sleep stage (e.g., a total time spent in each sleep stage during a sleep session or durations for each of the consecutive sleep stages that occur in the sleep session). In some cases, time spent in each sleep stage can be weighted, such as based on a usage variable. For example, the sleep quality score may be calculated with weighting values such that time spent in certain sleep stages while the user interface seal is above a threshold value has a greater impact on the sleep quality score than time spent in certain sleep stages while the user interface seal is below the threshold value.

In some cases, sleep quality score can be based at least in part on physiological data associated with the user, such as i) respiration rate; ii) heart rate; iii) heart rate variability; iv) movement data; v) electroencephalograph data; vi) blood oxygen saturation data; vii) respiration rate variability; viii) respiration depth; ix) tidal volume data; x) inspiration amplitude data; xi) expiration amplitude data; xii) inspiration volume data; xiii) expiration volume data; xiv) inspiration-expiration ratio data; xv) perspiration data; xvi) temperature data; xvii) pulse transit time data; xviii) blood pressure data; xix) position data; xx) posture data; xxi) blood sugar level data; or xxii) any combination of i-xxi.

In some cases, sleep stage information (and/or optionally a usage variable) can be used to remove or otherwise discount data from a particular usage variable. For example, if the event information is indicative of an event occurring at 2:01:43 AM, but the sleep stage information indicates that the user was not asleep at that time, that detected event can be removed or otherwise discounted from the event information usage variable.

The sleep performance score can be presented to a user in any suitable fashion, such as via a display device on a respiratory therapy device, a display device on a user device (e.g., a smartphone), or otherwise. Presentation of the sleep performance score can include presenting a total sleep performance score, as well as presenting one or more component scores that make up the entire sleep performance score. Component scores can be based on individual or combined scores for each of the usage variable(s), as well as sleep stage information and/or a sleep quality score. In some cases, presenting the sleep performance score can include presenting a graphical representation of the component scores that make up the sleep performance score.

In some cases, presenting the sleep performance score can include presenting component scores broken down and/or sorted by the level of contribution the component score provides to the sleep performance score. In some cases, such a breakdown or sorting can be associated with the weighting values used to calculate the sleep performance score. In an example, if usage time during REM sleep and event information during REM sleep are weighted highly, but user interface compliance information during wakefulness or light sleep is weighted lowly, presenting the sleep performance score may including indicating that usage time during REM sleep and event information during REM sleep were important components to this sleep session's sleep performance score, optionally indicating that user interface compliance information during wakefulness or light sleep was less important.

In some cases, presenting the sleep performance score can include presenting component scores (e.g., an amount of contribution to the sleep performance score) for one or more usage variables broken down (e.g., binned) and/or sorted by sleep stage information. For example, a set of four component scores (e.g., bins) may be presented for a usage time variable, including a score for usage time during wakefulness, a score for usage time during REM sleep, a score for usage time during light sleep, and a score for usage time during deep sleep. It should be understood that each of the component scores can be a score that is calculated by applying a weighting value to the usage variable as described herein with reference to calculating an overall sleep performance score.

The sleep performance score can act as an objective measurement of the user's sleep session. In some cases, the sleep performance score can be limited to only that portion of the user's sleep session during which respiratory therapy was used. The sleep performance score can provide information to the user to help monitor, maintain, and/or encourage compliance (e.g., use of the respiratory therapy device as desired or prescribed). In some cases, the sleep performances score can provide information to healthcare providers, facilities, and/or healthcare-related companies (e.g., healthcare insurance providers) about the compliance and efficacy of a user making use of the respiratory therapy device during sleep. In some cases, the sleep performance score can be used to provide objective measurements for research purposes.

In some cases, the sleep performance score can be used to influence or adjust parameters associated with a future use of the user's respiratory therapy system or another's respiratory therapy system. Such influence or adjustment can be manual (e.g., a user switching user interfaces) or automatic (e.g., a respiratory therapy device automatically altering air pressures supplied during use). In an example, after recording one or more sleep performance scores (e.g., for one or more sleep sessions), one or more additional sleep performance scores can be measured (e.g., for one or more additional sleep sessions) after one or more parameters of the respiratory therapy system are adjusted. Then, the additional sleep performance score(s) can be compared with the original sleep performance score(s) to determine if the adjustment(s) were beneficial or not. If the adjustments were not beneficial, they may be reverted. If the adjustments were beneficial, they may be kept for further use or adjusted further. In some cases, data associated with changes in sleep performance score as correlated to one or more adjustments of the respiratory therapy system can be transmitted to a server (e.g., a cloud-based or Internet-accessible server). Such data can be used in the production of future respiratory therapy systems and/or be accessed by existing respiratory therapy systems to improve respiratory therapy.

In some cases, the sleep performance score and/or a sleep quality sore can be used to identify one or more usage variables that are tolerated by the user, even if out of range. In some cases, in addition to calculating the sleep performance score (and/or sleep quality score), an out-of-range usage variable can be identified. Identifying an out-of-range usage variable can include determining that a value of the usage variable falls outside of a desired threshold range (e.g., below a threshold value, above a threshold value, or between two threshold values). An out-of-range usage variable can be one whose overall value is out of the desired threshold range (e.g., a count of the number of detected events in an event information variable being above a threshold number of events), one whose value is outside of the desired threshold range for a duration of time (e.g., having a seal quality variable below a threshold value for a threshold duration of total time during a sleep session), or one whose score is outside of a desired threshold range (e.g., a pre-weighted or post-weighted score, such as a component score). In some cases, if the sleep performance score (and/or sleep quality score) is above a threshold amount while one or more particular usage variables are out-of-range, for a single sleep session or for multiple sleep sessions (e.g., at least a threshold number of sleep sessions or a threshold number of consecutive sleep sessions), it can be determined that the given usage out-of-range variables may nonetheless be tolerated usage variables. In such cases, the tolerated usage variables may be deemed to be less important to overall sleep performance, sleep quality, and/or respiratory therapy effectiveness.

In an example, while a poor seal quality may normally be a problem that should be remedied (e.g., by replacing the user interface), if a particular user achieves high sleep performance scores (and/or sleep quality scores) despite having a poor seal quality (e.g., a seal quality variable below a threshold value), the respiratory therapy system can deem seal quality to be a tolerated variable. Once the seal quality is deemed a tolerated variable, the system can choose to not notify the user to change the user interface, can lower one or more weighting values associated with the seal quality variable, can make one or more adjustments to the respiratory therapy system, can take other actions related to the seal quality variable, or any combination thereof.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

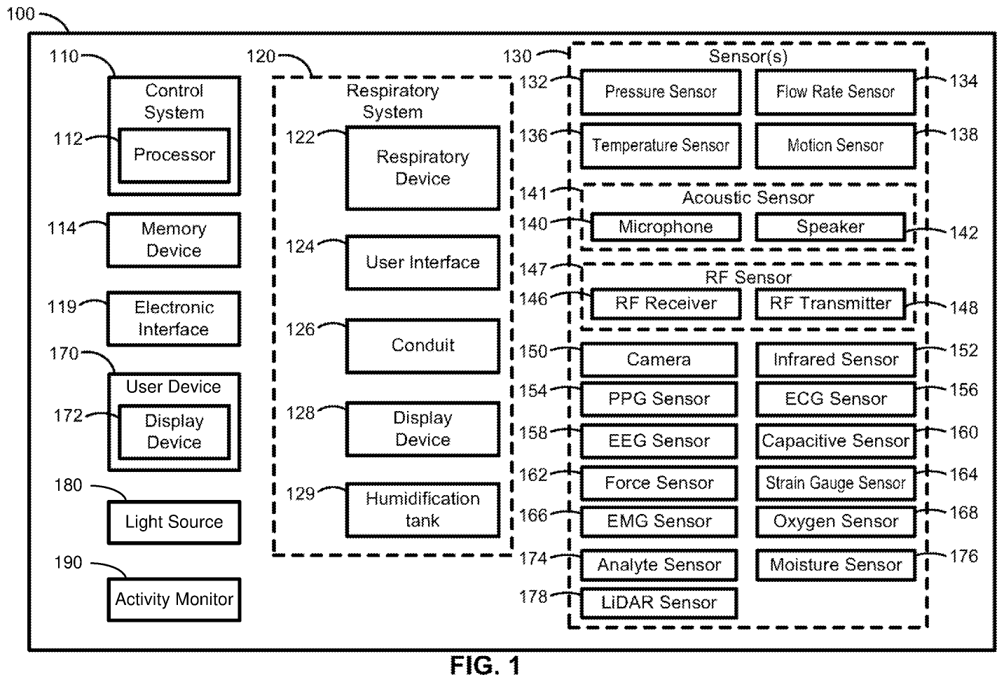

Referring to FIG. 1, a system 100, according to some implementations of the present disclosure, is illustrated. The system 100 includes a control system 110, a memory device 114, an electronic interface 119, a respiratory therapy system 120, one or more sensors 130, one or more user devices 170, one or more light sources 180, and one or more activity trackers 190.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, a portion (e.g., a housing) of the respiratory system 120, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia, an employment status of the user, an educational status of the user, a socioeconomic status of the user, or any combination thereof. The medical information can include, for example, including indicative of one or more medical conditions associated with the user, medication usage by the user, or both. The medical information data can further include a multiple sleep latency test (MSLT) test result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory system 120 (also referred to as a respiratory therapy system). The respiratory system 120 can include a respiratory pressure therapy device 122 (referred to herein as respiratory device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

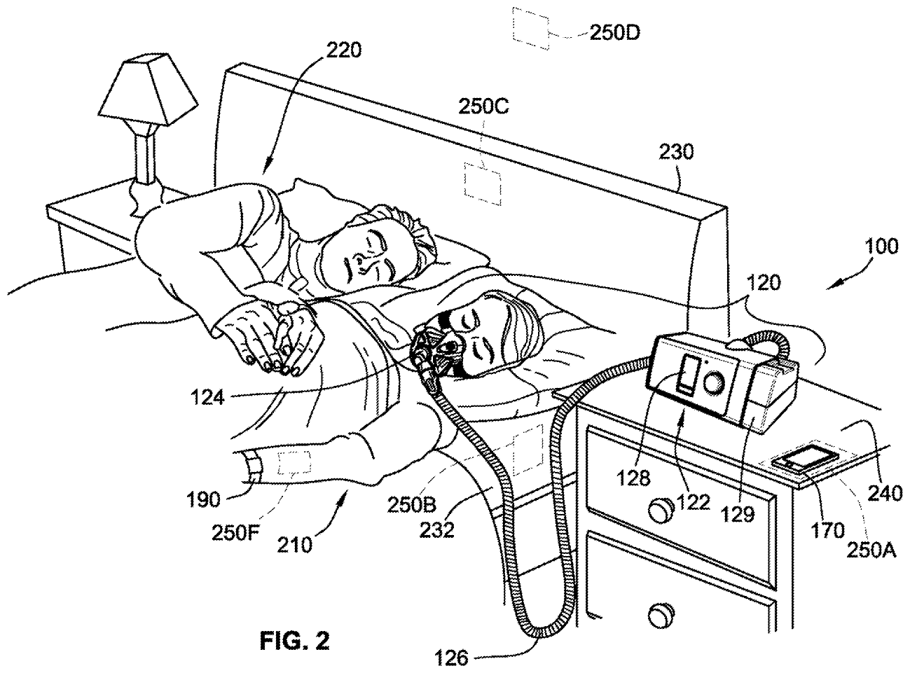

As shown in FIG. 2, in some implementations, the user interface 124 is a face mask that covers the nose and mouth of the user. Alternatively, the user interface 124 can be a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. In some examples, the user interface 124 can be a tube-up mask, wherein straps of the mask are configured to act as conduit(s) to deliver pressurized air to the face or nasal mask. The user interface 124 can also include one or more vents for permitting the escape of carbon dioxide and other gases exhaled by the user 210. In other implementations, the user interface 124 can comprise a mouthpiece (e.g., a night guard mouthpiece molded to conform to the user's teeth, a mandibular repositioning device, etc.).

The conduit 126 (also referred to as an air circuit or tube) allows the flow of air between two components of a respiratory system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep performance score, a sleep score or a therapy score (such as a myAir™ score), the current date/time, personal information for the user 210, etc.). In some implementations, the display device 128 acts as a human-machine interface (HI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122 and includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory system 120 and a bed partner 220 are located in a bed 230 and are laying on a mattress 232. The user interface 124 (e.g., a full face mask) can be worn by the user 210 during a sleep session. The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example, physiological data, audio data, or both. Physiological data generated by one or more of the sensors 130 can be used by the control system 110 to determine a sleep-wake signal associated with a user during a sleep session and one or more sleep-related parameters. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. N1 and N2 can be considered light sleep stages, whereas N3 can be considered a deep sleep stage. The sleep-wake signal can also be timestamped to indicate a time that the user enters the bed, a time that the user exits the bed, a time that the user attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the user during the sleep session based on the sleep-wake signal include a total time in bed, a total sleep time, a sleep onset latency, a wake-after-sleep-onset parameter, a sleep efficiency, a fragmentation index, or any combination thereof.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory system 120 and/or ambient pressure. In such implementations, the pressure sensor 132 can be coupled to or integrated in the respiratory device 122. The pressure sensor 132 can be, for example, a capacitive sensor, an electromagnetic sensor, a piezoelectric sensor, a strain-gauge sensor, an optical sensor, a potentiometric sensor, or any combination thereof. In one example, the pressure sensor 132 can be used to determine a blood pressure of a user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The microphone 140 outputs audio data that can be stored in the memory device 114 and/or analyzed by the processor 112 of the control system 110. The audio data generated by the microphone 140 is reproducible as one or more sound(s) during a sleep session (e.g., sounds from the user 210). The audio data form the microphone 140 can also be used to identify (e.g., using the control system 110) an event experienced by the user during the sleep session, as described in further detail herein. The microphone 140 can be coupled to or integrated in the respiratory device 122, the use interface 124, the conduit 126, or the user device 170.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory device 122, the user interface 124, the conduit 126, or the user device 170.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user 210 enters the bed 230 (FIG. 2), and to determine a time when the user 210 exits the bed 230.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a face mask that covers the nose and mouth of the user 210, the analyte sensor 174 can be positioned within the face mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the face mask (in implementations where the user interface 124 is a face mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be coupled to or integrated in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the user device 170, or any combination thereof. For example, the microphone 140 and speaker 142 is integrated in and/or coupled to the user device 170 and the pressure sensor 130 and/or flow rate sensor 132 are integrated in and/or coupled to the respiratory device 122. In some implementations, at least one of the one or more sensors 130 is not coupled to the respiratory device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

For example, as shown in FIG. 2, one or more of the sensors 130 can be located in a first position 250A on the nightstand 240 adjacent to the bed 230 and the user 210. Alternatively, one or more of the sensors 130 can be located in a second position 250B on and/or in the mattress 232 (e.g., the sensor is coupled to and/or integrated in the mattress 232). Further, one or more of the sensors 130 can be located in a third position 250C on the bed 230 (e.g., the secondary sensor(s) 140 is couple to and/or integrated in a headboard, a footboard, or other location on the frame of the bed 230). One or more of the sensors 130 can also be located in a fourth position 250D on a wall or ceiling that is generally adjacent to the bed 230 and/or the user 210. The one or more of the sensors 130 can also be located in a fifth position such that the one or more of the sensors 130 is coupled to and/or positioned on and/or inside a housing of the respiratory device 122 of the respiratory system 120. Further, one or more of the sensors 130 can be located in a sixth position 250F such that the sensor is coupled to and/or positioned on the user 210 (e.g., the sensor(s) is embedded in or coupled to fabric or clothing worn by the user 210 during the sleep session). More generally, the one or more of the sensors 130 can be positioned at any suitable location relative to the user 210 such that the sensor(s) 140 can generate physiological data associated with the user 210 and/or the bed partner 220 during one or more sleep session.

The user device 170 (FIG. 1) includes a display device 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The light source 180 is generally used to emit light having an intensity and a wavelength (e.g., color). For example, the light source 180 can emit light having a wavelength between about 380 nm and about 700 nm (e.g., a wavelength in the visible light spectrum). The light source 180 can include, for example, one or more light emitting diodes (LEDs), one or more organic light emitting diodes (OLEDs), a light bulb, a lamp, an incandescent light bulb, a CFL lightbulb, a halogen lightbulb, or any combination thereof. In some implementations, the intensity and/or wavelength (e.g., color) of light emitted from the light source 180 can be modified by the control system 110. The light source 180 can also emit light in a predetermined pattern of emission, such as, for example, continuous emission, pulsed emission, periodic emission of differing intensities (e.g., light emission cycles including a gradual increase in intensity followed by a decrease in intensity), or any combination thereof. Light emitted from the light source 180 can be viewed directly by the user or, alternatively, reflected or refracted prior to reaching the user. In some implementations, the light source 180 includes one or more light pipes.

In some implementations, the light source 180 is physically coupled to or integrated in the respiratory therapy system 120. For example, the light source 180 can be physically coupled to or integrated in the respiratory device 122, the user interface 124, the conduit 126, the display device 128, or any combination thereof. In some implementations, the light source 180 is physically coupled to or integrated in the user device 170. In other implementations, the light source 180 is separate and distinct from each of the respiratory therapy system 120 and the user device 170, and the activity tracker 190. In such implementations, the light source 180 can be positioned to the user 210 (FIG. 2), for example, on the nightstand 240, the bed 230, other furniture, a wall, a ceiling, etc.

The activity tracker 190 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the user, such as a smartwatch, a wristband, a ring, or a patch. For example, referring to FIG. 2, the activity tracker 190 is worn on a wrist of the user 210. The activity tracker 190 can also be coupled to or integrated a garment or clothing that is worn by the user. Alternatively still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory 114, the respiratory system 120, and/or the user device 170.

While the control system 110 and the memory device 114 are described and shown in FIG. 1 as being a separate and distinct component of the system 100, in some implementations, the control system 110 and/or the memory device 114 are integrated in the user device 170 and/or the respiratory device 122. Alternatively, in some implementations, the control system 110 or a portion thereof (e.g., the processor 112) can be located in a cloud (e.g., integrated in a server, integrated in an Internet of Things (IoT) device (e.g., a smart TV, a smart thermostat, a smart appliance, smart lighting, etc.), connected to the cloud, be subject to edge cloud processing, etc.), located in one or more servers (e.g., remote servers, local servers, etc., or any combination thereof.

While system 100 is shown as including all of the components described above, more or fewer components can be included in a system for generating physiological data and determining a recommended notification or action for the user according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory system 120, at least one of the one or more sensors 130, and the user device 170. Thus, various systems can be formed using any portion or portions of the components shown and described herein and/or in combination with one or more other components.

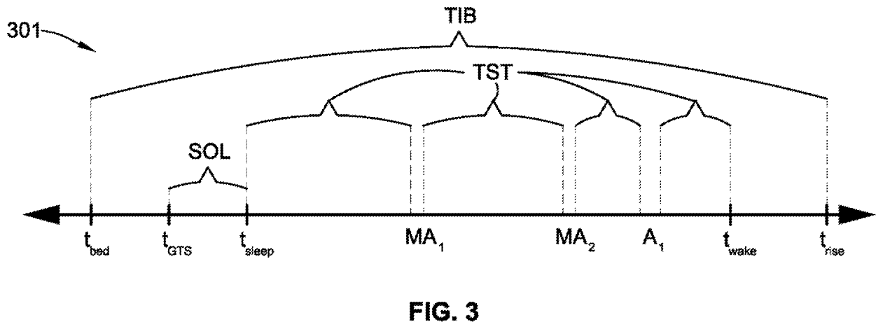

As used herein, a sleep session can be defined in a number of ways based on, for example, an initial start time and an end time. Referring to FIG. 3, an exemplary timeline 301 for a sleep session is illustrated. The timeline 301 includes an enter bed time (t_{bed}), a go-to-sleep time (t_{GTS}), an initial sleep time (tₛₗₑₑₚ), a first micro-awakening MA₁ and a second micro-awakening MA₂, a wake-up time (t_{wake}), and a rising time (tᵣᵢₛₑ).

In some implementations, a sleep session is a duration where the user is asleep. In such implementations, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) a user-selectable element that is displayed on the display device 172 of the user device 170 (FIG. 1) to manually initiate or terminate the sleep session.

The enter bed time t_{bed} is associated with the time that the user initially enters the bed (e.g., bed 230 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 301 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

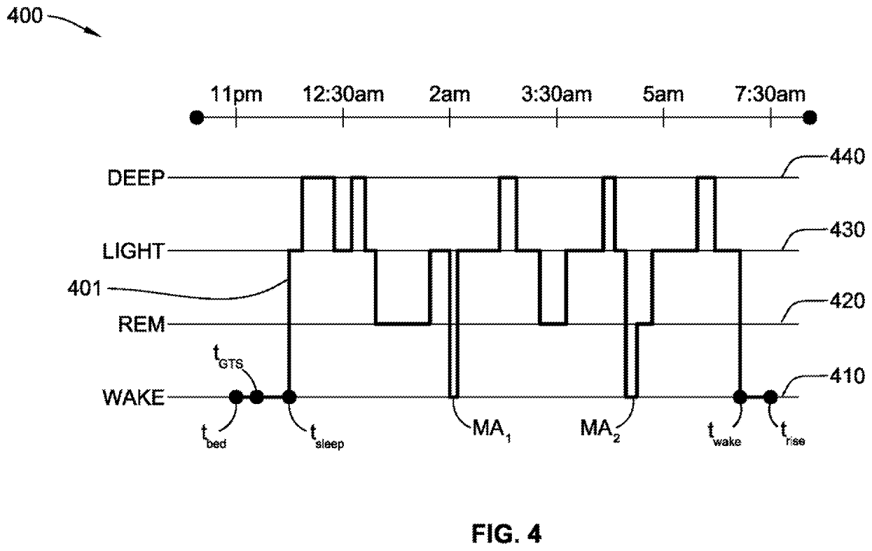

Referring to FIG. 4, an exemplary hypnogram 400 corresponding to the timeline 400 (FIG. 4), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410, 420, 430, 440 is indicative of the sleep stage at any given time during the sleep session.

The sleep-wake signal 401 can be generated based on physiological data associated with the user (e.g., generated by one or more of the sensors 130 (FIG. 1) described herein). The sleep-wake signal can be indicative of one or more sleep states or stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights), data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory device 122, data indicative of the user donning the user interface 124, etc.), or any combination thereof.

While hypnogram 400 depicts progressively shorter REM stages as the sleep session progresses, that is not always the case. In some cases, the duration of REM stages progressively increases as the sleep session progresses (e.g., with the first REM stage being shorter than the last REM stage).

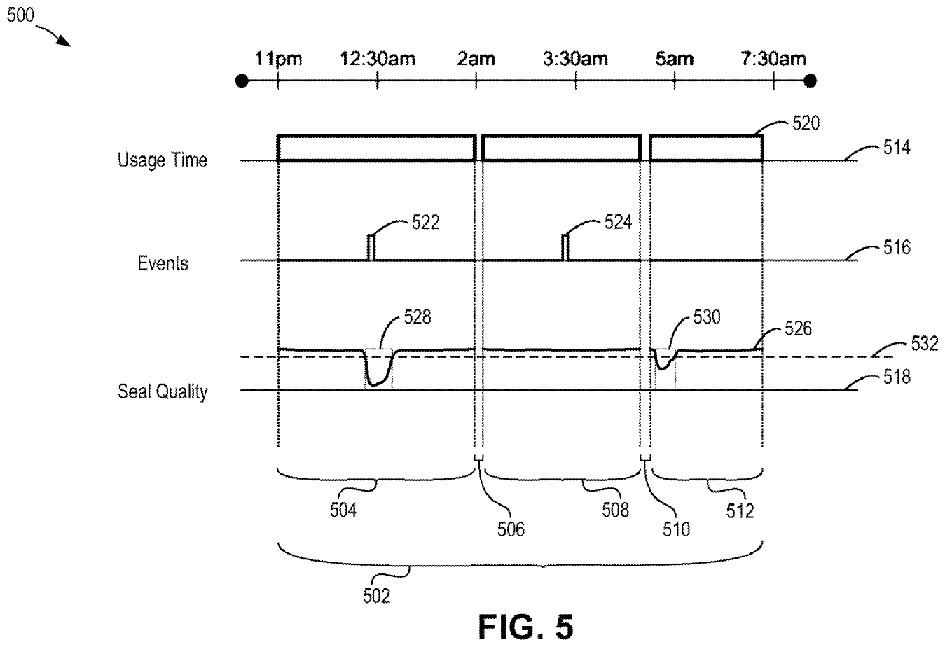

FIG. 5 is a chart 500 illustrating certain usage variables associated with the hypnogram of FIG. 4, according to certain aspects of the present disclosure. Chart 500 can be associated with the sleep session of FIG. 3. The chart 500 includes several usage variables, including usage time 514, events 516, and seal quality 518, as determined over the course of a sleep session 502. Additionally, a user interface compliance usage variable can be determined and/or shown based on the detected user interface transitions which appear as user interface transition periods 506, 510 (e.g., gaps in other usage variables). In some cases, the user interface compliance usage variable can be or can include one or more mask on-off events (e.g., events denoting donning or removal of a mask or user interface). In some cases, the user interface compliance usage variable can track when the user interface is put on and/or taken off, and/or how many times the user interface is put on and/or taken off

Usage time 514 can represent the amount of time the respiratory therapy system (e.g., respiratory therapy system 120 of FIG. 1) is used to provide respiratory therapy to the user. As depicted in chart 500, a set of blocks 520 are depicted across the sleep session for usage time 514, representing blocks of time during which the user was using the respiratory therapy system. For example, the respiratory therapy system was used during a first period 504, a second period 508, and a third period 512. Between the first period 504 and the second period 508, the user may have temporarily stopped using the respiratory therapy system (e.g., by removing and replacing the user interface) as identified by user interface transition period 506. The start of the user interface transition period 506 can indicate a first user interface transition (e.g., removal of the user interface), whereas the end of the user interface transition period 506 can indicate a second user interface transition (e.g., donning of the user interface). Likewise, a similar user interface transition period 510 is located between the second period 508 and the third period 512.

The events 516 usage variable can be represented as a collection of timestamped values (or merely timestamps), as indicated by event 522 and event 524 depicted in chart 500. Events 522, 524 can be apnea events, hypopnea events, or other events.

The seal quality 518 usage variable can be represented by a line 526 representing values associated with seal quality during the sleep session. In the example of seal quality 518, there are two instances 528, 530 of low seal quality, during which times line 526 had dropped below a threshold line 532. In some cases, user interface transition periods 506, 510 can be discounted for purposes of the seal quality 518 usage variable, or can be indicative of instances of low seal quality.

When comparing the usage variables depicted in chart 500 with the sleep stages depicted in hypnogram 400, one can see that the first period 504 included time from t_{bed} up to MA₁. Presumably, the user was making use of the respiratory therapy device during that time, only temporarily taking it off during MA_{I}. During that first period 504, the user passed through four stages of light sleep, two stages of deep sleep, and one stage of REM sleep. During that first period 504, a low seal quality instance 528 was detected, which coincided with a detected event 522. Presumably, the low seal quality at instance 528 may have resulted in insufficient respiratory therapy, thus permitting event 522 to occur. Event 522 can also coincide with the user temporarily dropping from a deep sleep stage into a light sleep stage.

Second period 508 shows usage time extending from the end of MA₁ up to the start of MA₂, which included four stages of light sleep, two stages of deep sleep, and one stage of REM sleep. During the second period 508, the seal quality 518 was shown as being strong (line 526 above threshold line 542) and one event 524 was detected. Comparing chart 500 with hypnogram 400, the event 524 occurred at approximately the same time the user was in the REM sleep stage.

Third period 512 shows usage time extending from the end of MA₂ up to t_{wake}, which included one REM sleep stage, two light sleep stages, and a single deep sleep stage. During the third period 512, no events were detected, but the third period 512 began with an instance 530 of low seal quality, which occurred during the REM sleep stage.

Because event 524 occurred in a REM sleep stage and event 522 occurred in a deep sleep stage, the occurrence of event 524 may be more highly weighted than the occurrence of event 522. For example, event 524 may reduce the overall sleep performance score more than event 522.

Because low seal quality instance 530 occurred in a REM sleep stage and low seal quality instance 528 occurred in light and deep sleep stages, the occurrence of low seal quality instance 530 may be more highly weighted than the occurrence of low seal quality instance 528. For example, low seal quality instance 530 may reduce the overall sleep performance score more than low seal quality instance 528.

Chart 500 shows one visual indication of an example set of usage variables that can be determined based on sensor data collected from one or more sensors (e.g., one or more sensors 130 of FIG. 1). Other sets of usage variables can include any combination of one or more of the usage variables disclosed herein or other similar usage variables associated with use of the respiratory therapy system. Additionally, any set of usage variables can be presented, stored, and/or otherwise represented in any suitable form, such as charts, numbers, spreadsheets, databases, strings of data, or other formats.

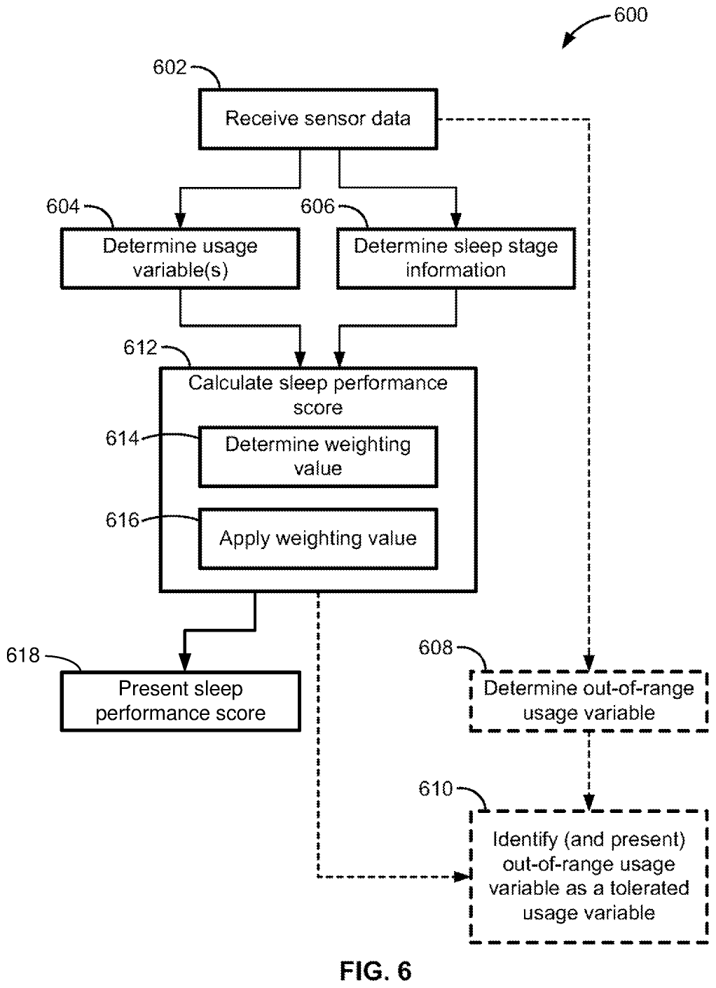

FIG. 6 is a flowchart depicting a process 600 for scoring sleep performance, according to certain aspects of the present disclosure. Process 600 can be carried out by any suitable system, such as system 100 of FIG. 1, including by processor 112 of control system 110 of FIG. 1. One, some, or all blocks of process 600 can occur during a sleep session (e.g., the given sleep session for which the sleep performance score is being calculated or a subsequent sleep session), immediately following a sleep session, or at another time. In some cases, process 600 is carried out by a user device (e.g., smartphone), such as user device 170 of FIG. 1.

At block 602, sensor data is received. The received sensor data can be collected from one or more sensors, such as one or more sensors associated with a sleep session of a user during which the user is receiving respiratory therapy from a respiratory therapy system (e.g., respiratory therapy system 120 of FIG. 1). Such one or more sensors (e.g., one or more sensors 130 of FIG. 1) can include a set of sensors of the respiratory therapy system (e.g., a pressure sensor and a flow rate sensor) and/or a set of sensors of a user device (e.g., an acoustic sensor or RF sensor of a smartphone), although other sensors can be used. In some cases, sensor data can be preprocessed prior to being received at block 602. In some cases, receiving sensor data at block 602 can include preprocessing the sensor data to improve the ability to later determine any usage variables and/or sleep stage information that may be desired. In some cases, no preprocessing is performed on the sensor data.

At block 604, one or more usage variables can be determined from the sensor data. Determining one or more usage variables can include processing the sensor data (e.g., via an equation, a function, or a machine learning algorithm) to identify one or more values for the one or more usage variables. The one or more usage variables can be any number or combination of suitable usage variables, such as those disclosed herein. In some cases, a usage variable determined at block 604 can be a single-value usage variable, such as an average leak flow rate, which can be represented as a single number, or a count of detected events, which can be indicated as a single number. In some cases, however, a usage variable determined at block 604 can be a set of values, such as timestamped values, or timestamps themselves, that occur throughout the sleep session. For example, a seal quality usage variable can be represented as a collection of seal quality values (e.g., 0-100%, 0-20 on a 20-point scale, or the like) collected periodically (e.g., based on a sampling rate).

At block 606, sleep stage information can be determined. Determining sleep stage information can include processing the sensor data to identify the sleep stage of the user at different points throughout the sleep session, such as to identify transitions between different sleep stages and durations of time spent in various sleep stages. Time spent in a sleep stage can refer to total time spent in all instances of a particular sleep stage (e.g., a total of 90 minutes of REM sleep throughout the sleep session) or time spent in individual instances of various sleep stages (e.g., a 40 minute REM stage followed by a 10 minute light sleep stage, followed by a 5 minute wakefulness stage (e.g., a microawakening), followed by a 30 minute light sleep stage, followed by a 10 minute deep stage, followed by a 15 minute light sleep stage, followed by another 20 minute REM stage). In some cases, sleep stage information can include duration of the entire sleep session. In some cases, sleep stage information can include one or more ratios between sleep stage durations and/or between each sleep stage duration and the duration of the total sleep session.

At block 612, a sleep performance score can be calculated. The sleep performance score can be calculated using the determined usage variable(s) from block 604 and the determined sleep stage information from block 606. In some cases, calculating the sleep performance score can include calculating one or more component scores that can be combined to calculate the final sleep performance score. In some cases, component scores can be determined for one, some, or all of the usage variables from block 604 and/or the sleep stage information determined at block 606.

In some cases, determining the sleep performance score at block 612 can include determining one or more weighting values at block 614 and applying the one or more weighting values at block 616. A weighting value can be determined for any combination of usage variables, sleep stage information, segmented usage variables, or segmented sleep stage information. In some cases, determining weighting values can include segmenting a usage variable into multiple usage variable segments. The segments can be based on sleep stages and/or other usage variables. For example, a usage time usage variable can be segmented based on sleep stages or an event information usage variable can be segmented based on a seal quality usage variable.

Determining a weighting value can include accessing a pre-defined weighting value, calculating a weighting value, or receiving the weighting value (e.g., receiving the weighting value from an output of a machine learning algorithm). In some cases, the determined weighting value can be a neutral weighting value, such as a 1.0× or 100% weighting value. In some cases, the determined weighting value can be an increasing weighting value, such as a 1.5× or 150% weighting value. In some cases, the determined weighting value can be a decreasing weighting value, such as a 0.5× or 50% weighting value.

In some cases, a weighting value for a usage variable can be determined based on the sleep stage information from block 606 and/or other usage variable(s) from block 604. In some cases, determining weighting values at block 614 can include determining a set of weighting values for the given usage variable, such as a weighting value for each combination of the given usage variable and the sleep stages from the sleep stage information and/or the other usage variables. In an example, weighting values determined for an event information usage variable (e.g., detected apnea or hypopnea events) can include determining 1) a weighting value for the event information usage variable in combination with a wakefulness sleep stage; 2) a weighting value for the event information usage variable in combination with a light sleep stage; 3) a weighting value for the event information usage variable in combination with a deep sleep stage; and 4) a weighting value for the event information usage variable in combination with an REM sleep stage.

In some cases, determining a weighting value for a given usage variable at block 604 can include applying another usage function (e.g., time-dependent usage variable) to a function. For example, a weighting value for a given usage variable can be a proportional or inverse proportional function of another usage variable.

In some cases, determining a weighting value can include accessing a database of weighting values. In some cases, accessing a database of weighting values can include using information associated with the user (e.g., physiological information and/or demographic information) to select one or more weighting values from the database of weighting values. For example, information associated with the user can be used to determine a population into which the user falls (e.g., based on an age range, gender information, geolocation, or the like) and then select one or more weighting values associated with the determined population. In some cases, health information (e.g., professional diagnoses, self-reported diagnoses, and/or health-related measurements) can be used to determine one or more weighting values.

Applying weighting values at block 616 can include applying one or more weighting values to one or more usage variables and/or sleep stage information. Applying a weighting value can include using the weighting value to calculate a component score for the usage variable and/or calculate a sub-component score for a segmented usage variable. In some cases, applying a weighting value can include multiplying the weighting value by the usage variable (or segmented usage variable or other such value). In some cases, applying weighting values at block 616 can include multiple weighting values to a given usage variable or usage variable segment. For example, a usage variable segment that is a usage time segment during REM sleep can have a first weighting value applied that is a weighting value calculated and/or selected specifically for usage time segments during REM sleep, as well as a second weighting value applied that is a weighting value calculated and/or selected globally for the usage variable and/or the sleep stage. For example, the first weighting value can be based on a preset weighting value and the second weighting value can be based on user information.

In some cases, calculating a sleep performance score at block 612 can be performed in other fashions while making use of the determined usage variable(s) from block 604 and the sleep stage information from block 606.

At block 618, the sleep performance score can be presented, such as to the user of the respiratory therapy system, a caregiver, or another entity. Presenting the sleep performance score can include presenting the sleep performance score in an easily digestible manner, such as a number (e.g., a number from 0 to 100), a percentage (e.g., a percentage from 0% to 100%), a color coded indicator, a graphical indicator (e.g., a bar or circular gauge filled according to the sleep performance score), or other such manner.

In some cases, presenting the sleep performance score at block 618 can further include presenting additional information, such as by default and/or upon receiving a trigger action (e.g., pressing of a button). In some cases, the additional information can include one or more component scores or sub-component scores. In some cases, the additional information can include a hypnogram of the sleep stage information. In some cases, the additional information can include a summary of sleep stage information and/or a summary of one or more component scores or sub-component scores. In some cases, the additional information can include an indication of how much a component score or sub-component score contributed to the sleep performance score. In some cases, the additional information can include a recommendation for making an adjustment to the respiratory therapy system for improving the sleep performance score. For example, the recommendation can include an instruction to replace a user interface or adjust a setting on the respiratory therapy device. In some cases, the additional information can include trend data indicating a trend in sleep performance score for the given sleep session and a number of preceding sleep sessions.

In some optional cases, an out-of-range usage variable can be determined at block 608. Determining an out-of-range usage variable can be based on the sensor data received from block 602. Determining an out-of-range usage variable can be separate from and/or part of determining usage variable(s) at block 604, and can include identifying that a value of the given usage variable is out of a threshold range (e.g., below a threshold level, above a threshold level, and/or between a lower threshold level and an upper threshold level).

At optional block 610, an out-of-range usage variable can be identified as a tolerated usage variable based on the calculated sleep performance score from block 612 and the out-of-range usage variable determined from block 608. The out-of-range usage variable can be identified as a tolerated usage variable when the sleep performance score is nevertheless above a threshold value. Thus, despite the given usage variable being out of a desired range, the sleep performance score still indicates a good sleep session with use of respiratory therapy (e.g., a sleep session with high quality and/or a sleep session with efficient and/or effective use of respiratory therapy). In some cases, identifying an out-of-range usage variable as a tolerated usage variable at block 610 can further include presenting the out-of-range usage variable as a tolerated usage variable (e.g., presenting an indication that a given usage variable is well-tolerated).

In some cases, once a usage variable is identified as a tolerated usage variable, future instances of determining weighting values at block 614 can include determining an adjusted weighting value for any usage variable identified as a tolerated usage variable. The adjusted weighting value can de-emphasize the effect of the tolerated usage variable on the sleep performance score. For example, if a user well-tolerates decreases in seal quality, calculation of future sleep performance scores can apply lower weighting values to the seal quality variable.

The blocks of process 600 can be performed in any suitable order, including certain blocks being performed simultaneously. For example, calculating sleep performance score at block 612 can occur simultaneously to determining an out-of-range usage variable. In another example, determining sleep stage information can occur after determining usage variable(s). Additionally, while process 600 is described with certain blocks, one, some, or all of the blocks of process 600 can be removed and/or replaced with other blocks. Additionally, in some cases, process 600 can include additional blocks not depicted in FIG. 6. For example, in some cases, calculating a sleep performance score at block 612 can further include determining a sleep quality score, as disclosed in further detail herein.

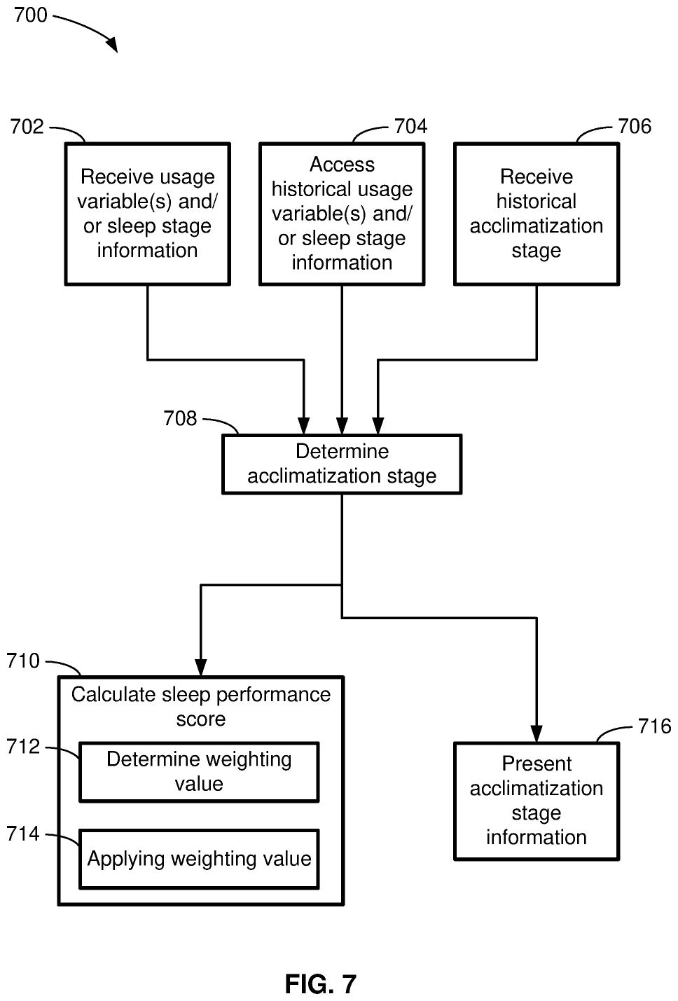

FIG. 7 is a flowchart depicting a process 700 for scoring sleep performance using acclimatization stages, according to certain aspects of the present disclosure. Process 700 can be carried out by any suitable system, such as system 100 of FIG. 1, including by processor 112 of control system 110 of FIG. 1. One, some, or all blocks of process 700 can occur during a sleep session (e.g., the given sleep session for which the sleep performance score is being calculated or a subsequent sleep session), immediately following a sleep session, or at another time. In some cases, process 700 is carried out by a user device (e.g., smartphone), such as user device 170 of FIG. 1. In some cases, some or all of process 700 can be performed as part of calculating a sleep performance score as described with reference to block 612 of FIG. 6.

Process 700 involves determining an acclimatization stage at block 708 and calculating a sleep performance score using the acclimatization stage at block 710 and/or presenting the acclimatization stage at block 708. Each acclimatization stages can modify how the sleep performance score is otherwise calculated and/or otherwise encourage the user to achieve a certain goal. Each acclimatization stage can be a stage with a distinct purpose. For example, an early acclimatization stage can be a stage designed to encourage the user to fall asleep while using therapy; a middle acclimatization stage can be a stage designed to encourage the user to sleep for longer while using therapy; a late acclimatization stage can be a stage designed to encourage the user to achieve a good sleep overall while using therapy; and a maintenance acclimatization stage can be stage designed to encourage the user to maintain a good sleep overall while using therapy.

Possible acclimatization stages can be established in a sequence, such as starting with the early acclimatization stage, moving next to a middle acclimatization stage, moving next to a late acclimatization stage, and then moving next to a maintenance acclimatization stage. For descriptive purposes, the acclimatization stages can be described vertically, starting with the early acclimatization stage at the bottom and moving up until reaching the maintenance acclimatization stage at the top. Any number of acclimatization stages can be used, such as two, three, four, or more than four. In some cases, non-sequential acclimatization stages can be used. For example, a set of possible acclimatization stages can include starting with an early acclimatization stage and ending with a maintenance acclimatization stage, but having a number of different, potential middle acclimatization stages that may be used depending on a user's circumstances. In such an example, the user may begin in an early acclimatization stage, move to a time-on-therapy middle acclimatization phase, then move to a total-sleep-time middle acclimatization phase, then move to a late acclimatization phase and then a maintenance acclimatization phase. While it is desired for a user to move sequentially through the acclimatization stages, in some cases it is possible for a user to move backwards to a previous acclimatization stage, such as if certain usage variables and/or sleep stage information shows the user's sleep is degrading or not improving sufficiently or shows the user is not engaging in therapy.

In some cases, determining an acclimatization stage at block 702 can include using information received at block 702. At block 702, one or more usage variables are received and/or sleep stage information is received. Receiving a usage variable can include determining the usage variable, such as described with reference to block 604 of FIG. 6. Receiving sleep stage information can include determining sleep stage information, such as described with reference to block 606 of FIG. 6. Using the information received at block 702, an acclimatization stage can be determined at block 708 based at least in part on the usage variable(s) and/or the sleep stage information. For example, in some cases the acclimatization stage can be based on whether or not the user achieves a sleep onset latency at or below a threshold time. A user achieving a longer sleep onset latency may be placed into an early acclimatization phase until they are able to achieve a shorter sleep onset latency. Any usage variable information and/or sleep stage information can be used in the determination of the acclimatization stage. In some cases, the determination of acclimatization stage can be based on achieving one or more desired threshold values for one or more usage variables for a threshold duration of time (e.g., achieving an average leak flow rate below a threshold value for at least 120 minutes or for at least 50% of the sleep session).

In some cases, determining the acclimatization stage at block 708 is based at least in part on historical usage variable(s) and/or historical sleep stage information accessed at block 704. This historical data received at block 704 can be usage variable(s) and/or sleep stage information associated with one or more sleep sessions prior to the current sleep sessions, such as historical data associated with the past set number of days (e.g., past 7 days or past 30 days), the past number of sleep sessions during which therapy was used, or the like. By analyzing the historical usage variable(s) and/or historical sleep stage information (e.g., identifying that one or more usage variable(s) or sleep stages met or exceeded a threshold value for a threshold duration of time), a determination can be made as to which acclimatization stage to use. For example, if the number of sleep sessions of available data fall below a threshold number (e.g., only two nights of data are available), a default acclimatization stage (e.g., an early acclimatization stage) can be used. If the historical data shows the user has achieved certain qualifying sleep achievements, such as a sleep onset latency of at or less than thirty minutes for at least three consecutive days, the acclimatization stage can be determined to be a different acclimatization stage (e.g., a middle acclimatization stage). Likewise, if the user's sleep onset latency is shown to be at or more than thirty minutes for at least three consecutive days, the acclimatization stage can be determined to be a different acclimatization stage that emphasizes sleep onset latency (e.g., an early acclimatization stage).

In some cases, determining the acclimatization stage at block 708 can be based at least in part on one or more historical acclimatization stages received at block 706. Receiving a historical acclimatization stage at block 706 can include receiving a current acclimatization stage (e.g., the acclimatization stage last determined for the user). Depending on the current and/or historical data received at blocks 702 and/or 704, respectively, the determination can be made at block 708 to keep the current acclimatization stage or move the user to a new acclimatization stage (e.g., move up from a middle acclimatization stage to a late acclimatization stage, or move down from a middle acclimatization stage to an early acclimatization stage). Thus, in some cases determining an acclimatization stage 708 can include i) using a default (e.g., initial) acclimatization stage; ii) moving to a sequentially next acclimatization stage from the current acclimatization stage; or iii) moving to a sequentially previous acclimatization stage from the current acclimatization stage.

In some cases, determining an acclimatization stage at block 708 can include determining an acclimatization score. The acclimatization score can be based on one or more desired values for one or more usage variables and/or for certain sleep stage information. As the user approaches the desired values, the acclimatization score can increase. Once the user achieves or exceeds the desired values, the acclimatization score can meet or exceed a threshold score indicating that the sequentially next acclimatization stages should be implemented. In some cases, each acclimatization stage includes its own set of desired values for one or more usage variables and/or for certain sleep stage information. For example, in an early acclimatization stage, the acclimatization score can be based on the user's sleep onset latency and average leak flow rate. As sleep onset latency and average leak flow rate decrease, the acclimatization score can increase. Once the user achieves a sufficiently low sleep onset latency (e.g., below 30 minutes) and a sufficiently low average leak flow rate (e.g., no leak or at most an acceptable level of leak), the acclimatization score can meet or exceed the threshold score necessary to move to a new acclimatization stage (e.g., to a middle acclimatization stage). In the middle acclimatization stage, the acclimatization score can be based on the user's sleep onset latency and total sleep time, and time on therapy. In the late acclimatization stage, the acclimatization score can be based on the user's sleep onset latency, total sleep time, time in different sleep stages, heart/respiration rate while asleep, and the like, as well as other therapy-related usage variables. In the maintenance acclimatization stage, the acclimatization score can be based on the same or similar usage variables and sleep stage information to that of the late acclimatization stage, but with a different weighting across the usage variables and sleep stage information.

In some cases, the determined acclimatization stage is used in the calculation of the sleep performance score at block 710. Calculating the sleep performance score at block 710 can be the same as or similar to calculating the sleep performance score at block 612 of FIG. 6, except with the use of acclimatization stages. In some cases, calculating the sleep performance score can include modifying the sleep performance score based on the determined acclimatization stage from block 708, such as by directly modifying the score based on the acclimatization score or by modifying the weighting values used for the sleep performance score based on the determined acclimatization stage.

For example, at block 712, a set of weighting values can be determined based at least in part on the determined acclimatization stage. As each determined acclimatization stage can emphasize different aspects of sleep and/or sleep therapy, different acclimatization stages can have different associated sets of weighting values. For example, an early acclimatization stage can be associated with a first set of weighting values that emphasize sleep onset latency and/or average leak flow rate; a middle acclimatization stage can be associated with a second set of weighting values that emphasize sleep onset latency, total sleep time, and time on therapy; a late acclimatization stage can be associated with a third set of weighting values that emphasize sleep onset latency, total sleep time, time in one or more select sleep stages (e.g., time in REM sleep and time in deep sleep), heart rate, respiration rate, and/or other usage variables; and a maintenance acclimatization stage can be associated with a fourth set of weighting values designed to encourage maintaining a sleep quality score at or above a threshold sleep quality score. Determining weighting values at block 712 can also take into account aspects of determining weighting values associated with block 614 of FIG. 6.

After weighting values are determined at block 712, the weighting values can be applied at block 714 to calculate a sleep performance score. Applying the weighting values at block 714 can be the same or similar to applying the weighting values at block 616 of FIG. 6.

Thus, in some cases, each acclimatization stage can affect how the sleep performance score is calculated while the user is in that acclimatization stage.

Additionally, or instead of calculating the sleep performance score at block 710, the acclimatization stage information can be presented at block 716, such as by being presented to a user or a third party monitoring the user (e.g., a caregiver or healthcare provider). Presenting the acclimatization stage information can include i) presenting which acclimatization stage the user is in (e.g., “You are in the middle acclimatization stage!”); ii) presenting an acclimatization score (e.g., “78%” or “78 out of 100” or “78”); iii) presenting a recommendation associated with the acclimatization stage (e.g., “Try to use your therapy for as long as possible tonight” for an early acclimatization stage and “You are doing great; don't forget to clean the tubing each week” for a late acclimatization stage or maintenance acclimatization stage); iv) presenting a requirement to move to the next acclimatization stage (e.g., “You have been going to sleep within 30 minutes for the past five nights. After two more nights, you will move to the next stage”); or v) any combination of i-iv.

In some cases, presenting acclimatization stage information at block 716 only occurs when the acclimatization stage determined at block 708 is different than an immediately prior acclimatization stage (e.g., “Congratulations, you are doing great at falling asleep while wearing your therapy device” for a movement from an early acclimatization stage to a middle acclimatization stage, or “It seems like your therapy device has been leaking for the past few nights; let's try to work on improving it” for a movement from a middle acclimatization stage to an early acclimatization stage).

The blocks of process 700 can be performed in any suitable order, including certain blocks being performed simultaneously. Additionally, while process 700 is described with certain blocks, one, some, or all of the blocks of process 700 can be removed and/or replaced with other blocks. Additionally, in some cases, process 700 can include additional blocks not depicted in FIG. 7.

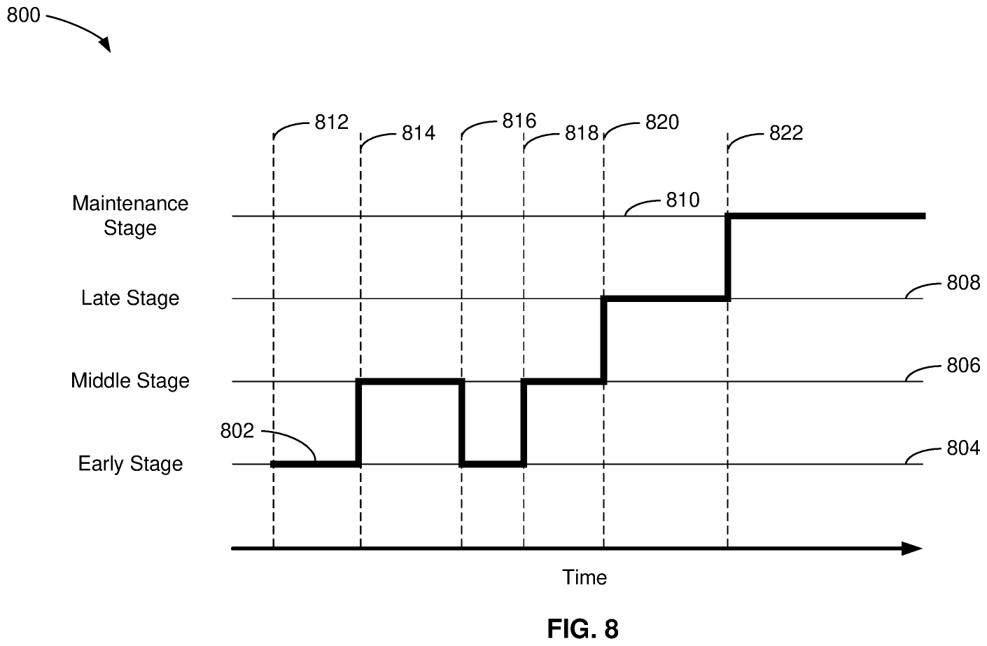

FIG. 8 is a chart 800 illustrating a user's progress through acclimatization stages, according to certain aspects of the present disclosure. Chart 800 depicts four acclimatization stages, including an early stage 804, a middle stage 806, a late stage 808, and a maintenance stage 810. The acclimatization sages depicted in chart 800 can be acclimatization stages as determined and leveraged with respect to process 700 of FIG. 7.

Line 802 represents the user's current acclimatization stage over time. The time axis represents the user engaging in multiple sleep sessions over the course of multiple days (e.g., over the course of 30 or 60 days).

On day 812, the user may first begin therapy. When first beginning therapy, the user may be automatically placed into the early stage 804.

On day 814, the user may have achieved several days of qualifying sleep achievements associated with the early stage 804 (e.g., sleep onset latency less than 30 minutes and less than a threshold level or leak), causing the user to be moved to the middle stage 806.

On day 816, however, the user may have achieved one or more days of poor sleep achievements (e.g., unacceptably high level of leak) that causes the user to move back to the early stage 804. By day 818, the user has once again achieved a sufficient number of days of qualifying sleep achievements, moving the user to the middle stage 806.

On day 820, the user may have achieved several days of new qualifying sleep achievements associated with the middle stage 806 (e.g., sleep onset latency at or below a threshold level, total sleep time above a threshold duration, and time on therapy above a threshold duration), permitting the user to move to the late stage 808.

On day 822, the user may have achieved several days of new qualifying sleep achievements associated with the late stage 808, permitting the user to move to the maintenance stage 810. The user may stay in the maintenance stage 810 thereafter. In some cases, the use of acclimatization stages can cease entirely after the user stays in the maintenance stage 810 for a threshold duration.

In some cases, a user may degrade to a previous acclimatization stage, such as depicted on day 816 with movement from the middle stage 806 to the early stage 804, however that need not always be the case. In some cases, acclimatization stages can be established to only proceed sequentially. In such cases, even despite poor sleep achievements, the user may remain in the same stage until qualifying for the next (e.g., the user would have remained in middle stage 806 from day 814 through day 816 and day 818 until qualifying for the late stage 808 on day 820).

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the present disclosure. Thus, the breadth and scope of the present disclosure should not be limited by any of the above described embodiments.

Although certain aspects of the present disclosure have been illustrated and described with respect to one or more implementations, equivalent alterations and modifications will occur or be known to others skilled in the art upon the reading and understanding of this specification and the annexed drawings. In addition, while a particular feature of an aspect of the present disclosure may have been disclosed with respect to only one of several implementations, such feature may be combined with one or more other features of the other implementations as may be desired and advantageous for any given or particular application.

One or more elements or aspects or steps, or any portion(s) thereof, from one or more of any of claims 1 to 35 below can be combined with one or more elements or aspects or steps, or any portion(s) thereof, from one or more of any of the other claims 1 to 35 or combinations thereof, to form one or more additional implementations and/or claims of the present disclosure.

What is claimed is:

1. A method for scoring sleep performance comprising:
receiving sensor data from one or more sensors, the sensor data being associated with a sleep session of a user using a respiratory therapy system;
determining, from the received sensor data, one or more usage variables associated with use of the respiratory therapy system;
determining, from the received sensor data, sleep stage information associated with the sleep session; and
calculating a sleep performance score for the sleep session based at least in part on the determined one or more usage variables and the sleep stage information.

2. The method of claim 1, wherein the one or more usage variables include:
i) usage time indicative of a duration of time the respiratory therapy system was used during the sleep session;
ii) a seal quality variable indicative of a quality of seal between the user and a user interface of the respiratory therapy system during use of the respiratory therapy system;
iii) event information indicative of a number of detected events that occurred during the sleep session;
iv) user interface compliance information associated with a number of detected user interface transition events in which the user interface was donned or removed during the sleep session; or
v) any combination of i-iv.

3. The method of claim 2, wherein the event information is indicative of a number of apnea-hypopnea events detected during the sleep session.

4. The method of any one of claims 1 to 3, wherein calculating the sleep performance score includes:
determining, for each of the one or more usage variables, a weighting value based at least in part on the sleep stage information; and
applying, for each usage variable of the one or more usage variables, the weighting value associated with the usage variable.

5. The method of any one of claims 1 to 4, wherein determining the one or more usage variables includes determining a usage time indicative of a duration of time the respiratory therapy system was used during the sleep session, wherein the sleep stage information is indicative of durations of time spent in a plurality of sleep stages, and wherein calculating the sleep performance score includes:
segmenting the determined usage time into a plurality of usage time segments based at least in part on the sleep stage information, wherein each of the usage time segments is associated with one of the plurality of sleep stages;
determining a usage time weighting value for each of the plurality of sleep stages; and
applying, to each of the usage time segments, the usage time weighting value associated with the respective sleep stage that is associated with the respective usage time segment.

6. The method of any one of claims 1 to 5, wherein determining the one or more usage variables includes determining a seal quality variable indicative of a quality of seal between the user and a user interface of the respiratory therapy system during use of the respiratory therapy system, wherein the sleep stage information is indicative of times spent in a plurality of sleep stages, and wherein calculating the sleep performance score includes:
segmenting the determined seal quality variable into a plurality of seal quality segments based at least in part on the sleep stage information, wherein each of the seal quality segments is associated with one of the plurality of sleep stages;
determining a seal quality weighting value for each of the plurality of sleep stages; and
applying, to each of the seal quality segments, the seal quality weighting value associated with the respective sleep stage that is associated with the respective seal quality segment.

7. The method of any one of claims 1 to 6, wherein determining the one or more usage variables includes determining event information indicative of a number of detected events that occurred during the sleep session, wherein the sleep stage information is indicative of times spent in a plurality of sleep stages, and wherein calculating the sleep performance score includes:
assigning, to each detected event of the event information and based at least in part on the sleep stage information, one of the plurality of sleep stages that coincides with a time of detection of the respective detected event;
determining an event weighting value for each of the plurality of sleep stages; and
applying, to each detected event of the event information, the event weighting value associated with the respective sleep stage that is associated with the respective detected event.

8. The method of any one of claims 1 to 7, wherein determining the one or more usage variables includes determining user interface compliance information associated with a number of detected user interface transition events in which the user interface was donned or removed during the sleep session, wherein the sleep stage information is indicative of times spent in a plurality of sleep stages, and wherein calculating the sleep performance score includes:
assigning, to each detected user interface transition event of the user interface compliance information and based at least in part on the sleep stage information, one of the plurality of sleep stages that coincides with a time of detection of the respective detected user interface transition event;
determining a user interface transition event weighting value for each of the plurality of sleep stages; and
applying, to each detected user interface transition event of the user interface compliance information, the user interface transition event weighting value associated with the respective sleep stage that is associated with the respective detected user interface transition event.

9. The method of any one of claims 1 to 8, further comprising determining a sleep quality score associated with the sleep session, wherein determining the sleep quality score is based at least in part on the sleep stage information.

10. The method of claim 9, wherein the sleep stage information is indicative of durations of time spent in a plurality of sleep stages, and wherein determining the sleep quality score includes:
segmenting the sleep stage information into sleep stage segments based at least in part on the one or more usage variables;
determining, for each of the sleep stage segments, a usage weighting value;
applying, within each of the sleep stage segments, the respective usage weighting value for the respective sleep stage segment to each sleep stage within the respective sleep stage segment.

11. The method of claim 9, wherein receiving the sensor data includes receiving physiological data associated with the user, and wherein determining the sleep quality score is further based at least in part on the received physiological data.

12. The method of claim 11, wherein the physiological data includes i) respiration rate; ii) heart rate; iii) heart rate variability; iv) movement data; v) electroencephalograph data; vi) blood oxygen saturation data; vii) respiration rate variability; viii) respiration depth; ix) tidal volume data; x) inspiration amplitude data; xi) expiration amplitude data; xii) inspiration volume data; xiii) expiration volume data; xiv) inspiration-expiration ratio data; xv) perspiration data; xvi) temperature data; xvii) pulse transit time data; xviii) blood pressure data; xix) position data; xx) posture data; xxi) blood sugar level data; or xxii) any combination of i-xxi.

13. The method of any one of claims 9 to 12, wherein calculating the sleep performance score for the sleep session is based at least in part on the sleep quality score.

14. The method of claim 13, wherein calculating the sleep performance score based at least in part on the sleep quality score includes applying one or more weightings to the determined one or more usage variables based at least in part on the sleep quality score.

15. The method of any one of claims 9-14, further comprising receiving user feedback associated with the sleep session, wherein calculating the sleep performance score based at least in part on the sleep quality score includes applying one or more weightings to the sleep quality score based at least in part on the user feedback.

16. The method of any one of claims 1 to 15, further comprising receiving user feedback associated with the sleep session, wherein calculating the sleep performance score based at least in part on the determined one or more usage variables includes applying one or more weightings to the determined one or more usage variables based at least in part on the user feedback.

17. The method of any one of claims 1 to 16, further comprising:
receiving user feedback associated with the sleep session;
determining a modification value based at least in part on the received user feedback; and
updating the sleep performance score by incorporating the modification value to the sleep performance score.

18. The method of any one of claims 1 to 17, wherein the one or more usage variables includes a first usage variable and a second usage variable, wherein calculating the sleep performance score based at least in part on the determined one or more usage variables includes applying a weighting to the first usage variable based at least in part on the second usage variable.

19. The method of claim 18, wherein applying the weighting to the first usage variable based at least in part on the second usage variable includes:
identifying a plurality of ranges associated with the second usage variable;
segmenting the first variable into a plurality of first usage variable segments based at least in part on the second usage variable, wherein each of the first usage variable segments is associated with one of the plurality of ranges associated with the second usage variable;
determining a weighting value for each of the plurality of ranges; and
applying, to each of the plurality of first usage variable segments, the weighting value associated with the respective one of the plurality of ranges associated with the respective first usage variable segment.

20. The method of any one of claims 1 to 19, wherein the one or more usage variables include:
i) average leak flow rate for the sleep session;
ii) a number of therapy sub-sessions within the sleep session;
iii) an average user interface pressure for the sleep session;
iv) a statistical summary of another of the one or more usage variables; or
v) any combination of i-iv.

21. The method of any one of claims 1 to 20, wherein the one or more usage variables includes event information indicative of a number of detected events that occurred during the sleep session, wherein calculating the sleep performance score based at least in part on the determined one or more usage variables and the sleep stage information includes:
identifying time periods in which the user was not asleep during the sleep session based at least in part on the sleep stage information; and
removing any detected events from the event information that occurred when the user was not asleep.

22. The method of any one of claims 1 to 21, further comprising:
identifying an out-of-range usage variable out of the one or more usage variables, wherein the out-of-range usage variable is outside of a desired threshold range;
identifying the sleep performance score as being above a sleep performance threshold; and
presenting an indication that the identified out-of-range usage variable is a tolerated usage variable.

23. The method of any one of claims 1 to 22, further comprising presenting the sleep performance score after completion of the sleep session.

24. The method of claim 23, wherein the sleep stage information is indicative of duration of time spent in a plurality of sleep stages, and wherein presenting the sleep performance score includes presenting total contribution to the sleep performance for each of the one or more usage variables, wherein presenting the total contribution for a given usage variable of the one or more usage variables includes presenting a plurality of sub-contributions binned by sleep stage for the given usage variable.

25. The method of any one of claims 1 to 24, wherein calculating the sleep performance score for the sleep session includes calculating the sleep performance score for only the portion of the sleep session coinciding with use of the respiratory therapy system.

26. The method of any one of claims 1 to 25, further comprising:
determining an acclimatization stage associated with the sleep session;
determining, for each of the one or more usage variables, a weighting value based at least in part on the acclimatization stage; and
applying, for each usage variable of the one or more usage variables, the weighting value associated with the usage variable.

27. The method of claim 26, wherein determining the acclimatization stage includes:
accessing (i) one or more historical usage variables associated with one or more historical sleep sessions of the user, (ii) historical sleep stage information associated with the one or more historical sleep sessions of the user, or (iii) both (i) and (ii); and
identifying the acclimatization stage based at least in part on (i) the one or more historical usage variables, (ii) the historical sleep stage information, or (iii) both (i) and (ii).

28. The method of any one of claim 26 or 27, further comprising accessing a historical acclimatization stage associated with the user, wherein determining the acclimatization stage is based at least in part on the historical acclimatization stage.

29. The method of any one of claims 26 to 28, wherein determining the acclimatization stage includes:
calculating an acclimatization score based at least in part on (i) the one or more usage variables, (ii) the sleep stage information, or (iii) both (i) and (ii); and
determining that the acclimatization score exceeds a threshold score associated with the acclimatization stage.

30. The method of any one of claims 26 to 29, wherein determining the acclimatization stage includes selecting the acclimatization stage from a set of possible acclimatization stages, wherein the set of possible acclimatization stages includes i) an early acclimatization stage, wherein the weighting values are a first set of weighting values that emphasize a sleep onset latency; ii) a middle acclimatization stage, wherein the weighting values are a second set of weighting values that emphasize a total sleep time; iii) a late acclimatization stage, wherein the weighting values are a third set of weighting values that emphasize a duration of time in one or more sleep stages; or
iv) any combination of i-iii.

31. The method of claim 30, wherein the set of possible acclimatization stages further includes a maintenance acclimatization stage, wherein the weighting values are a fourth set of weighting values associated with maintaining a sleep quality score at or above a threshold sleep quality score.

32. A system comprising:
a control system including one or more processors; and
a memory having stored thereon machine readable instructions;
wherein the control system is coupled to the memory, and the method of any one of claims 1 to 31 is implemented when the machine executable instructions in the memory are executed by at least one of the one or more processors of the control system.

33. A system for scoring sleep performance, the system including a control system configured to implement the method of any one of claims 1 to 31.

34. A computer program product comprising instructions which, when executed by a computer, cause the computer to carry out the method of any one of claims 1 to 31.

35. The computer program product of claim 34, wherein the computer program product is a non-transitory computer readable medium.
